(19) United States
(12) Patent Application Publication
(10) Pub. No.: US 2008/0071064 A1
(43) Pub. Date: Mar. 20, 2008

(54) TEMPERING

(76) Inventors: Torsten SCHULTZ-FADEMRECHT, Maselheim (DE); Patrick GARIDEL, Norderstedt (DE); Beate FISCHER, Bad Waldsee (DE); Karoline BECHTOLD-PETERS, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/766,925

(22) Filed: Jun. 22, 2007

Related U.S. Application Data
(60) Provisional application No. 60/806,691, filed on Jul. 6, 2006.

(30) Foreign Application Priority Data
Jun. 29, 2006 (DE) 10 2006 030 166.8

(51) Int. Cl. C07K 16/00

(52) U.S. Cl. 530/387.1

(57) ABSTRACT

The invention relates to the controlled crystallisation of powders, particularly spray-dried powders, for improving the flowability and the aerodynamic characteristics thereof and a method of reducing the electrostatics of a powder.

This application claims priority benefit from German application DE 10 2006 030 166.8, filed Jun. 29, 2006, and from U.S. provisional application Ser. No. 60/806,691, filed Jul. 6, 2006, the contents of which are incorporated herein by reference in their entireties.

### BACKGROUND TO THE INVENTION

1. Technical Field

The invention relates to a method of controlled crystallisation of powder, particularly spray-dried powder. Furthermore the invention relates to a method of increasing, maintaining or minimising the reduction in the flowability (FPF) of a powder, particularly while retaining the stability of the substance, a method of improving the aerodynamic properties of a powder and a method of better filling a powder, particularly a spray-dried powder.

2. Background

Various strategies are used to optimise the flowability of powders. On the one hand, the roughness of the particle surface may be increased. On the other hand, however, it is also possible to modify the chemical composition of the surface. Both by the increased roughness and by the chemical modification of the particle surface, interparticle interactions can be reduced, thus improving the flowability of the powders and also the dispersibility of the particles in air and hence the aerodynamic characteristics.

The roughness may be increased for example by coating the particles with nano-scale particles. (G. Huber, Powder Technology 134 (2003), 181-192, Electrostatically supported surface coating of a solid particle in liquid nitrogen for the use in Dry-Powder-Inhalers). Conventional methods of applying nanoparticles to powders (without focussing on spray-dried material) include for example mechanical methods such as e.g. coating in a jet grinder or in a hybridizer (Messrs Nara). Moreover, gravity mixers are also used (M. Eber, 2004, Dissertation Uni Erlangen, entitled: Wirksamkeit und Leistungsfähigkeit von Nanoskaligen Flussregulierungsmitteln [Action and Effectiveness of Nano-scale Flow Regulators}). When mixing spray-dried material with carrier systems sieves or gravity mixers are normally used.

Besides the strategy of modifying the surface roughness, the powder qualities may also be optimised by rendering the particle surface hydrophobic. When preparing spray-dried powders hydrophobic substances may be added directly to the spray solution. Both by atomising the spray solution into tiny droplets and during the evaporation of the drops in the drying tower of the spray dryer, the hydrophobic substances accumulate on the surface, as a result of the lower solubility of the excipient compared with the active substance and further excipients.

It is also possible to coat the spray-dried particles with a hydrophobic film in a separate step.

As a rule the aim with powders, particularly protein-containing powders and most particularly spray-dried powders, is to obtain the particles in amorphous form, as uncontrolled crystallisation processes can damage the active substance. Usually amorphous powders are hygroscopic and have a tendency to form powder agglomerates. Both effects are essentially undesirable and impose additional demands in terms of the storage of the powders and their delivery, for example when they are administered to the lungs.

With high protein contents in the spray-dried powders these powders also have a tendency to clumping. Depending on the protein more or less serious sticking together of the individual particles takes place. Whereas for example human serum albumin can be satisfactorily spray-dried at mass contents in excess of 70%, the product quality often suffers in the case of monoclonal antibodies. The resulting powders exhibit poor flowability and are difficult to disperse using an inhaler.

This gives rise to the challenge, for the product developer, of both achieving stability, particularly protein stability after spray-drying, and also producing a powder which is both free-flowing and also suitable for inhalation.

The state of the art in the solution to this problem is to carry out a series of process steps one after another. The literature describes the coating of spray-dried particles with so-called film forming agents or the mixing of spray-dried particles with further excipients, for example with nanoscale particles, or also with substantially larger particles measuring approx. 50-100 μm.

When coating materials, particularly spray-dried materials, with nanoparticles or with film-forming agents, such as e.g. Mg-stearate, high expenditure on equipments is essential. The use of grinding mills also causes thermal stress on the particles, so that unwanted morphological changes and damage to the substance, particularly the protein, may occur.

All processes that including mixing operations are critical particularly with regard to the homogeneity of the active substance in the powder and hence in terms of the uniformity of the dose. Inhomogeneities may occur directly during manufacture, but also during subsequent storage as a result of segregation. For example, during storage, an active substance may accumulate in the primary packing such as capsules or blisters. When mixing particles of different density, separation processes may occur as a result of gravity. When processing amorphous powders it is essential in multi-stage processes to drive the process chain at reduced humidity levels throughout, as otherwise uncontrolled crystallisation processes may occur. This may lead to higher costs in the process development and also in the manufacture of a product.

The problem is thus to solve the problems stated at reduced technical cost.

The problem on which the invention is based is solved by the following embodiments and by the objects and methods recited in the claims.

The present invention relates to a method of increasing, maintaining or minimising the reduction in the flowability (FPF) of a powder, a method of improving the aerodynamic properties of a powder and a method of reducing the electrostatics of a powder containing an active substance, particularly a protein, and at least one excipient, characterised in that
an amorphous powder is exposed
over a defined exposure period
in controlled manner to a water-containing gas or a solvent-containing gas with a defined relative humidity at a defined temperature.

The present invention preferably relates to methods according to the invention wherein the exposure period is selected such that the excipient crystallises before the active substance.

In a particularly preferred embodiment the powder in question is a spray-dried powder.

This procedure or this method is hereinafter also referred to as “tempering”. The tempering produces a thermodynamically stable particle surface. This reduces the extent of unwanted temperature- and humidity-induced changes in the powders during storage.

The homogeneity of the active substance in the powder is not critical in so far as it results from the composition of the spray droplet. Separation processes are impossible or unknown with purely spray-dried powders.

The tempering, besides conferring storage stability, may also optimise the flow and dispersion characteristics of the powders. Thanks to the thermodynamic stabilisation of the particle surface they may also be stored at higher humidities.

This improves the product safety, particularly for the patient. Producing a nanoscale surface roughness improves the flowability and the aerodynamics. This in turn is demonstrated by better filling/processing qualities and inhalability.

Applications for the present invention can be found for example in the development of powder-containing formulations of medicaments, e.g. for inhalation.

### SUMMARY OF THE INVENTION

Tempering creates a thermodynamically stable particle surface. As a result, the extent of unwanted temperature- and humidity-induced changes in the powders during storage is reduced. The homogeneity of the active substance in the powder is not critical in so far as it results from the composition of the spray droplet. Separation processes are impossible or unknown with purely spray-dried powders.

In conventional methods of preparing particularly protein-containing powders, uncontrolled crystallisation effects are avoided, as they could damage the powder or the protein. Surprisingly, however, it has been found that with certain recipes surface crystallisation can be induced without damaging the substance or the active substance, and particularly the protein.

The occurrence of surface crystallisation is associated with a number of preconditions: the powder, particularly the spray-dried powder, contains low-protein and high-protein areas. This zone formation may be caused by the use of substances of different degrees of hydrophobicity in the spray solution. The low-protein areas should contain substances which crystallise easily. The high-protein areas, on the other hand, should be considerably more difficult to crystallise and generally contain besides the protein another, third component, e.g. sugar. The easily crystallised substances should preferably be found on the particle surface; the substances that crystallise with difficulty, on the other hand, should be in the nucleus. The desired crystallisation of the particles should be controllable by humidity, temperature and time and takes place in a separate step, particularly after spray drying.

The additional mixing in of crystallisation inhibitors such as HSA may improve the particle properties of powders. Crystallisation inhibitors assist the formation of an amorphous matrix inside the particle nucleus where the readily water-soluble components, such as e.g. sugars and the protein are found.

The invention does not arise from the prior art.

Conventional methods, such as the process of applying nanoparticles to powders (without focussing on spray-dried material) are for example mechanical methods such as e.g. coating in a jet grinder or in a hybridizer (Messrs Nara). Moreover, gravity mixers are also used (M. Eber, 2004, Dissertation Uni Erlangen, entitled: Wirksamkeit und Leistungsfähigkeit von Nanoskaligen Flussregulierungsmitteln [Action and Effectiveness of Nanoscale Flow Regulators]). When mixing spray-dried material with carrier systems sieves or gravity mixers are normally used.

In one patent application (WO20040/3848) powders (including spray-dried powder) were mixed, after manufacture, with an amino acid, with Mg-stearate and with a phospholipid in a grinding mill (jet grinder/ball mill). However, there is no reference to a method of controlled crystallisation. The methods described in this patent application relate to rendering the particle surface hydrophobic. Thus, there was a description of how it was possible to reduce interparticulate interactions by this hydrophobic treatment and thereby optimise the flowability and the aerodynamic properties of the powders. However, the present invention does not relate to rendering the particle surface hydrophobic, but rather to thermodynamic stabilisation of the surface by controlled crystallisation. Another advantage of this method is the reduction in the electrostatic interactions in the powder. Powders rendered especially hydrophobic have a tendency to powerful electrostatic discharges. Thus, it was possible to demonstrate with phenylalanine-containing powders, for example, that the electrostatics was reduced after the tempering process.

Another patent application WO03/037303 also describes a method in which hydrophobic substances are applied directly to particles in the spray dryer. In this process, 2 spray solutions are fed independently of one another into the drying tower through a multiple nozzle. In one Example in the published patent application both raffinose and leucine particles are prepared. The particles are mixed directly in the spray dryer. The resulting mixture exhibited improved dispersion characteristics compared with the spray-dried raffinose. WO03/037303 is not relevant, as this method is concerned with the mixing of two spray-dried particle populations. This procedure however is not a part of the present invention. The present invention is concerned rather with modifying the existing particles without adding further substances in an additional process step.

In a further patent application (WO0030614) a process is described in which amorphous fractions are crystallised. The powder is acted upon by a supercritical or subcritical gas. The gas additionally contains water or an organic solvent. The supercritical or subcritical gas penetrates into the particle and by means of the solvent vapour causes the crystallisation of amorphous fractions. WO0030614 is not relevant, as the published application describes only supercritical methods. The present patent application however rules out supercritical methods in its preferred embodiment. The tempering of spray-dried particles essentially also comprises the controlled crystallisation of surfaces while retaining the amorphous fractions inside the particle. The protein can be stabilised by an amorphous environment. This essential step of the process is not a part of patent application WO0030614.

The U.S. Pat. No. 556,293, U.S. Pat. No. 5,709,884, U.S. Pat. No. 5,874,063 also describe processes in which powders are conditioned using solvent vapours. The vapour may consist both of water and of an organic solvent such as for example ethanol.

The U.S. Pat. No. 5,562,923 describes a method in which mechanically micronised particles are combined with solvent vapour, consisting of a low-chained alcohol or ketone or ethyl acetate. However, the U.S. Pat. No. 556,293 is not relevant, as proteins do not figure in the US patent. Moreover, according to the above-mentioned patent specification, only mechanically micronised powders are conditioned. Spray-dried powders also do not figure in U.S. Pat. No. 5,562,923.

The U.S. Pat. No. 570,984 is not relevant, as proteins do not figure in the US patent. Moreover, only powder mixtures consisting of different separately prepared substances or particles are conditioned, and not spray-dried powders.

The U.S. Pat. No. 5,874,063 is not relevant, as proteins do not figure in the US patent. Moreover the goal of this method is to almost totally reduce the amorphous fraction to crystalline particles. In the tempering of spray-dried powder the particle is substantially amorphous. This means that the crystallinity is less than 50%. After tempering, amorphous fractions are also needed for the protein stabilisation. This circumstance clearly restricts the present application/invention over U.S. Pat. No. 5,874,063.

Other spray-drying processes are described in the literature, which produce crystalline particles by a suitable choice of the spraying liquid.

Kambiz Gilani et al. (Journal of Pharmaceutical Science, Vol 94, No 5. 2005, page 1048-1059) showed that by adding ethanol to an aqueous spray solution the crystallinity of dried particles containing sodium cromoglycate could be increased. By increasing the crystalline fractions in the spray-dried particles it was also possible to improve the aerodynamic properties.

Harjunen et al. (Drug Development and Industrial Pharmacy, Vol 28, No. 8, 2002, Page 949-955) showed that by varying the mixing ratio of water and ethanol in a lactose-containing spray solution it is possible to prepare particles with amorphous fractions of between 0% and 100%.

However, these methods are not comparable with the controlled crystallisation of surfaces. For example, as described by Harjunen et al., lactose at 15% parts by weight in ethanol is present as a crystalline suspension. The spray drying is used here for solid/liquid separation and not for generating new particles.

### DESCRIPTION OF THE FIGURES

All the percentages mentioned in the descriptions refer to concentration data and compositions of the dry solids, particularly in a powder obtained by spray-drying (W/W).

FIG. 1

DVS (Dynamic Vapor Sorption)—photographs for determining the hygroscopicity of the spray-dried powder containing 80% phenylalanine, 10% LS90P and 10% IgG1

The Figure shows the hygroscopicity of a spray-dried powder. The measurement was carried out with a DVS (Messrs Porotec). The DVS method comprises weighing the sample and exposing the sample to water vapour under controlled conditions. The change in mass is detected. In this Figure, 2 cycles were run, each comprising steam adsorption and a corresponding desorption. The maximum relative humidity (RH) was 80%. By comparing the two cycles it is possible to detect humidity-induced irreversible results. In the present measurement a drop in mass can be detected both at 50% RH and at 60% RH. This drop results from the collapsing of the surface caused by crystallisation of the powder. As a result of the collapsing there is suddenly a supersaturation of condensed water vapour on the surface. This results in evaporation of this water and accordingly a reduction in mass.

FIG. 2

Hygroscopicity of a spray-dried powder containing 80% phenylalanine, 10% LS90P and 10% IgG1 at 50% relative humidity (RH) (FIG. 2a) and 60% RH (FIG. 2b)

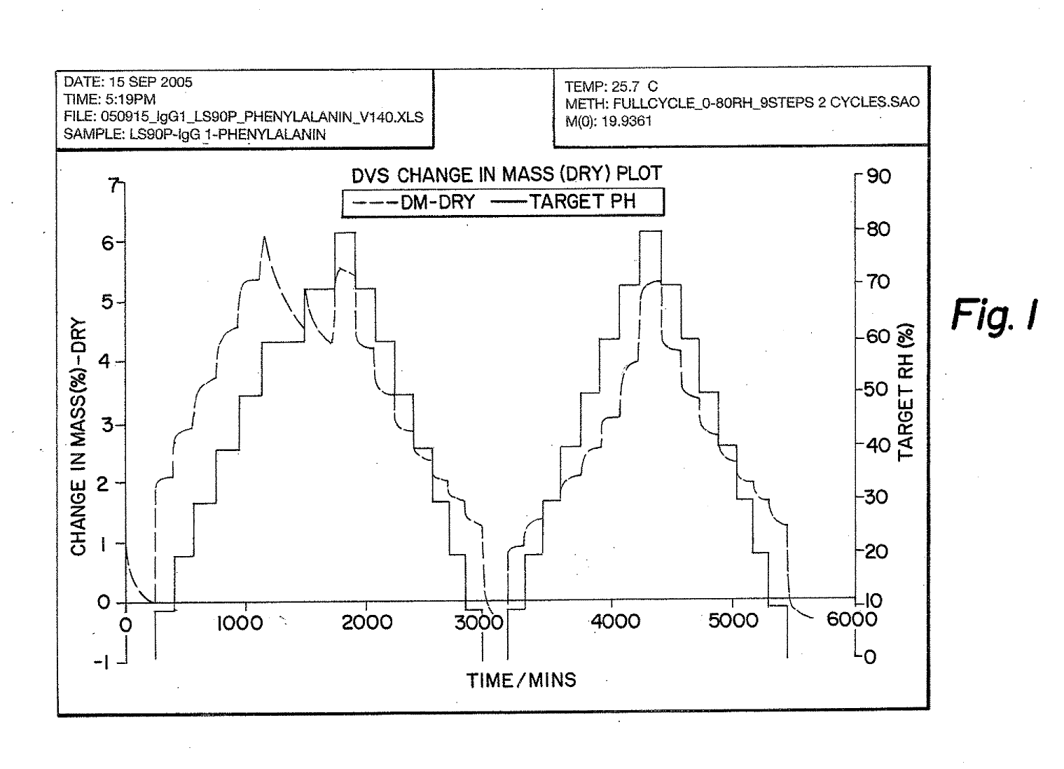

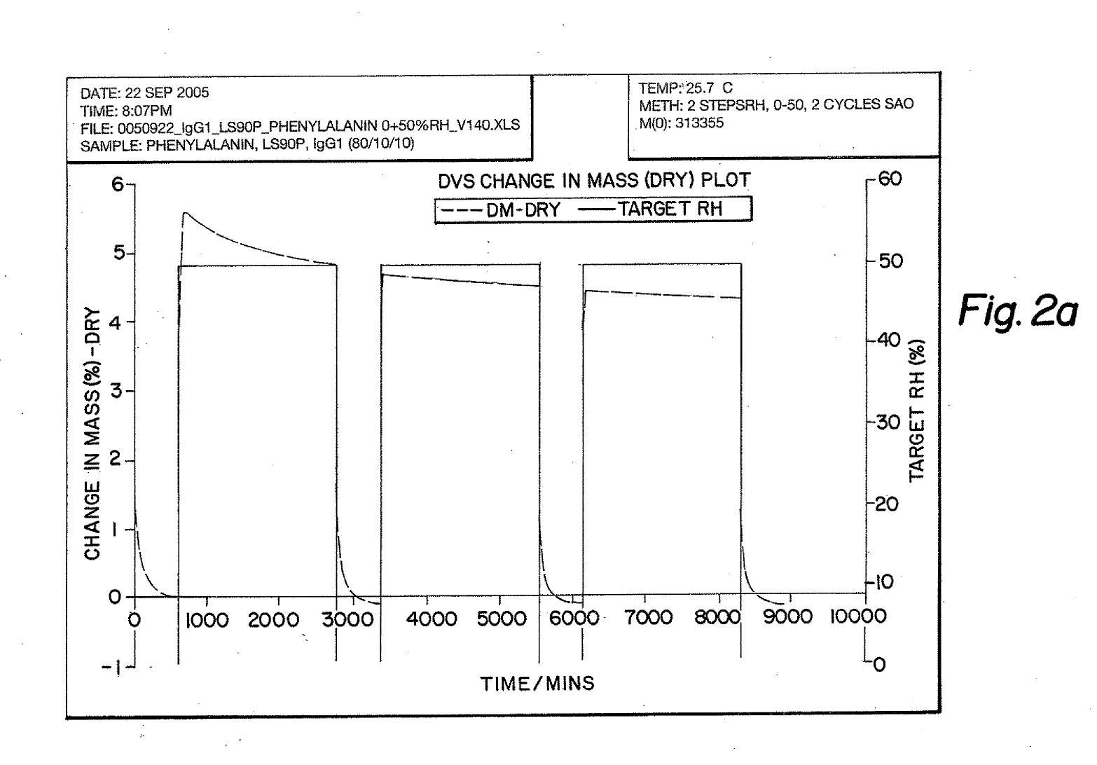

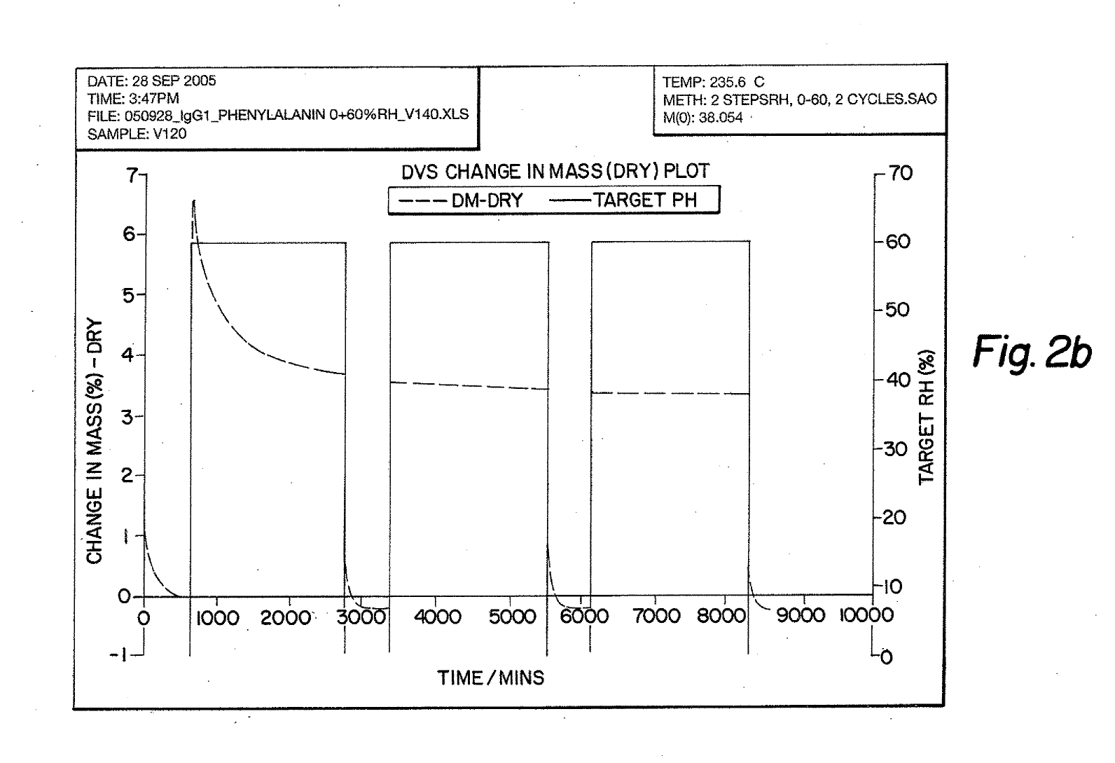

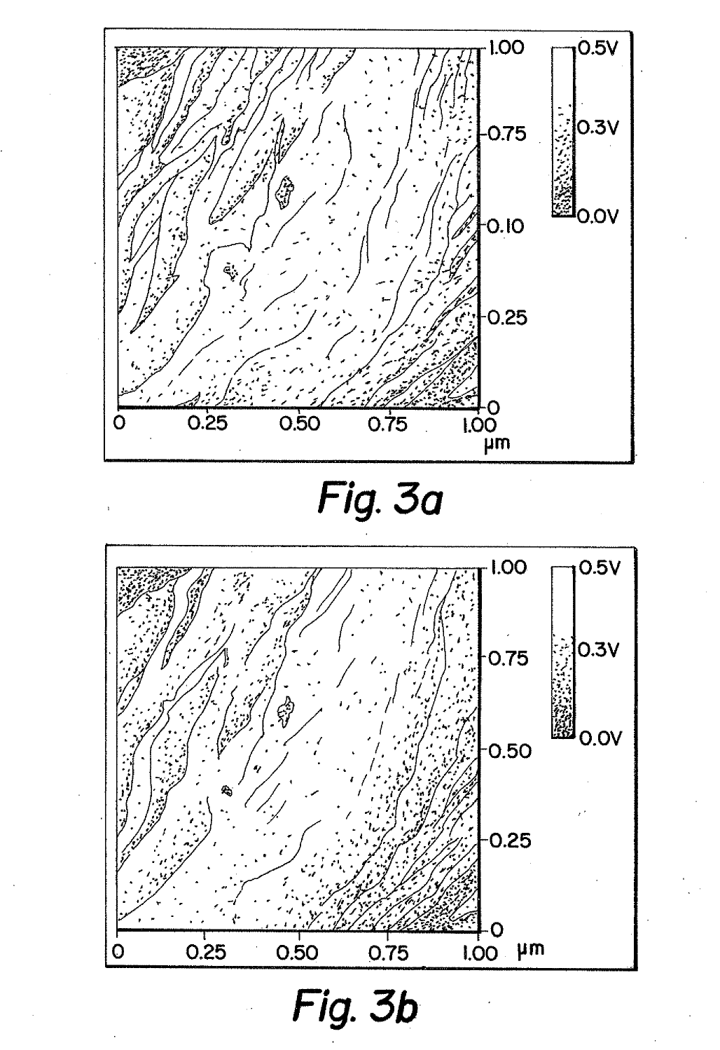

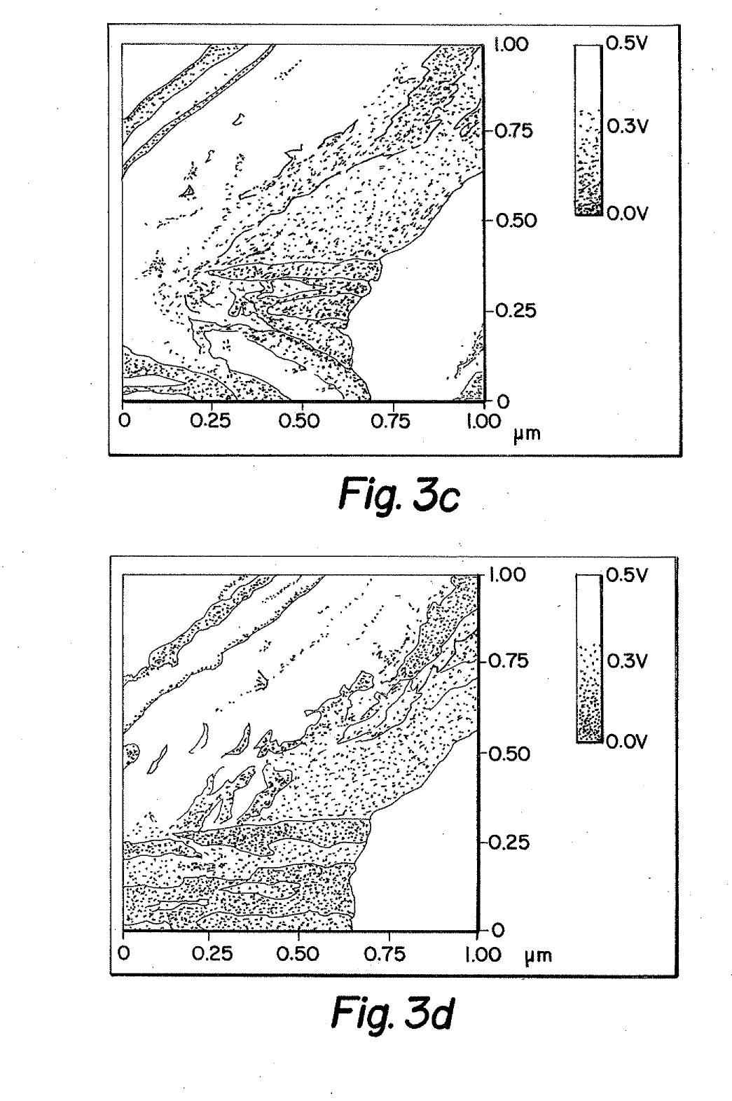

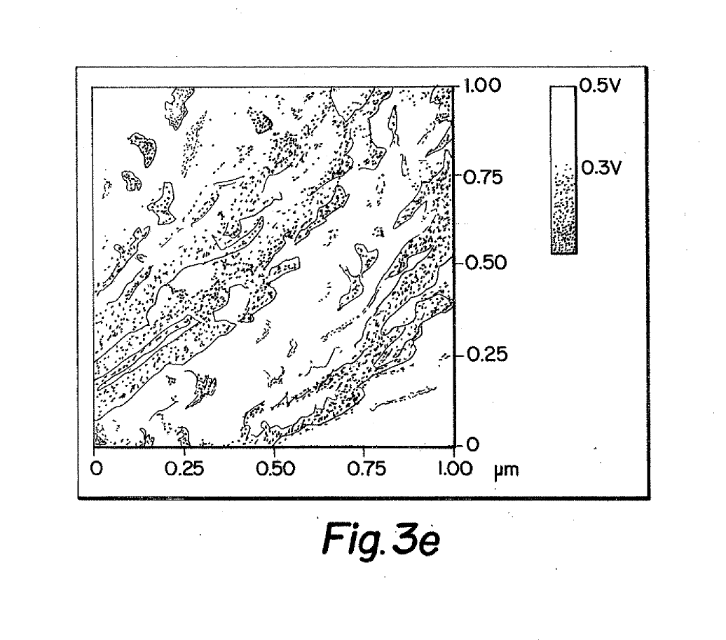

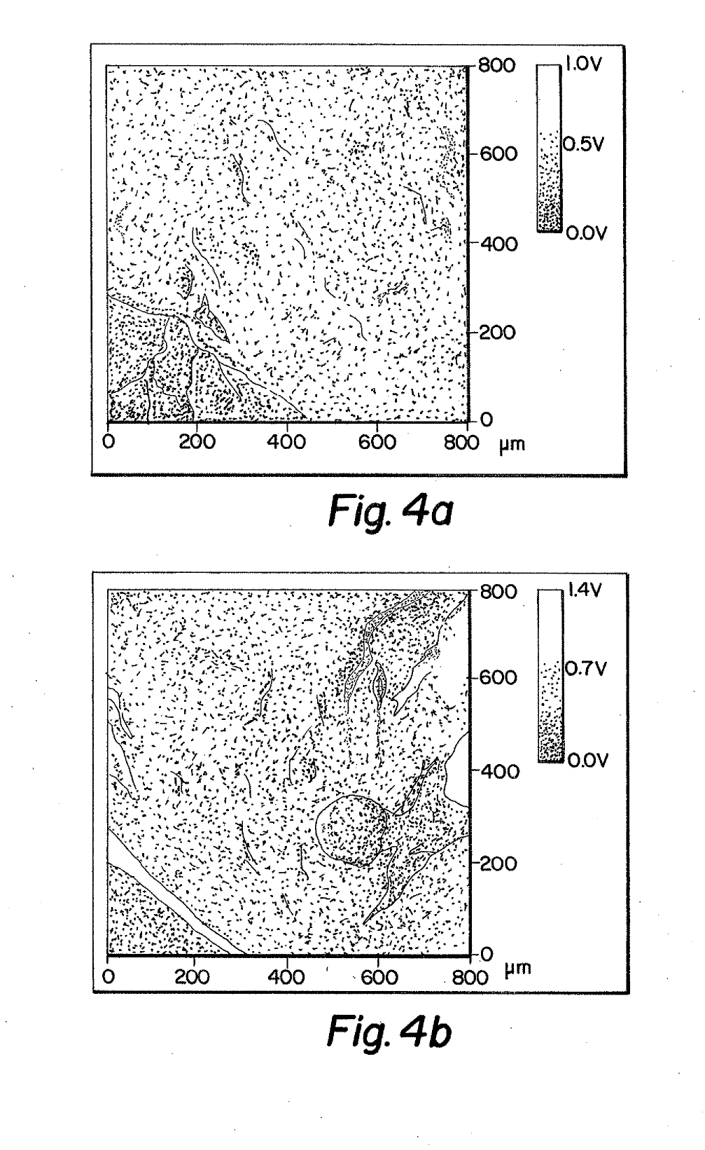

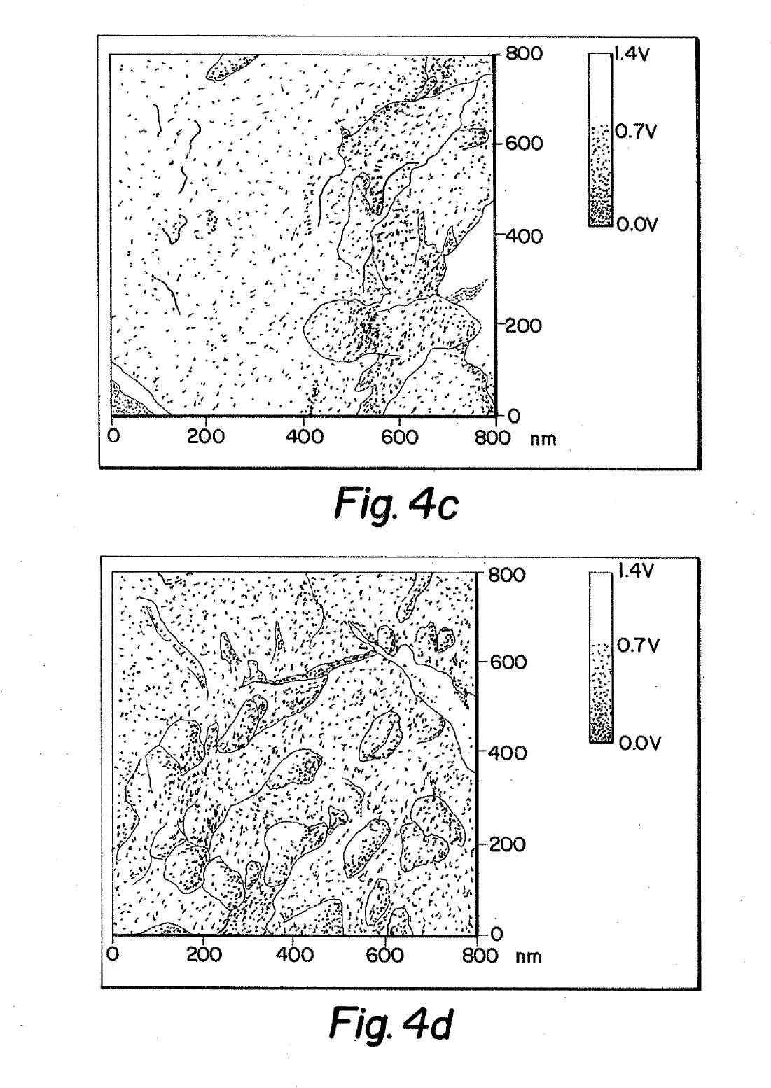

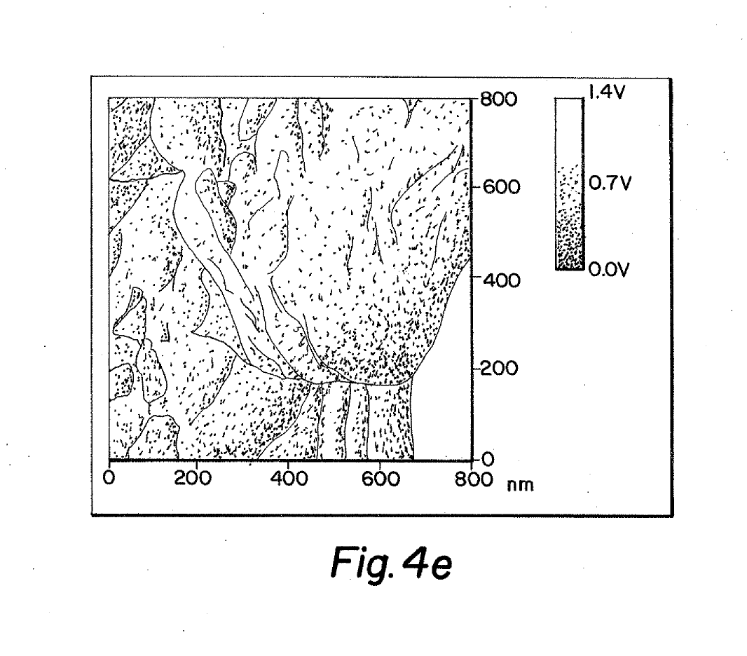

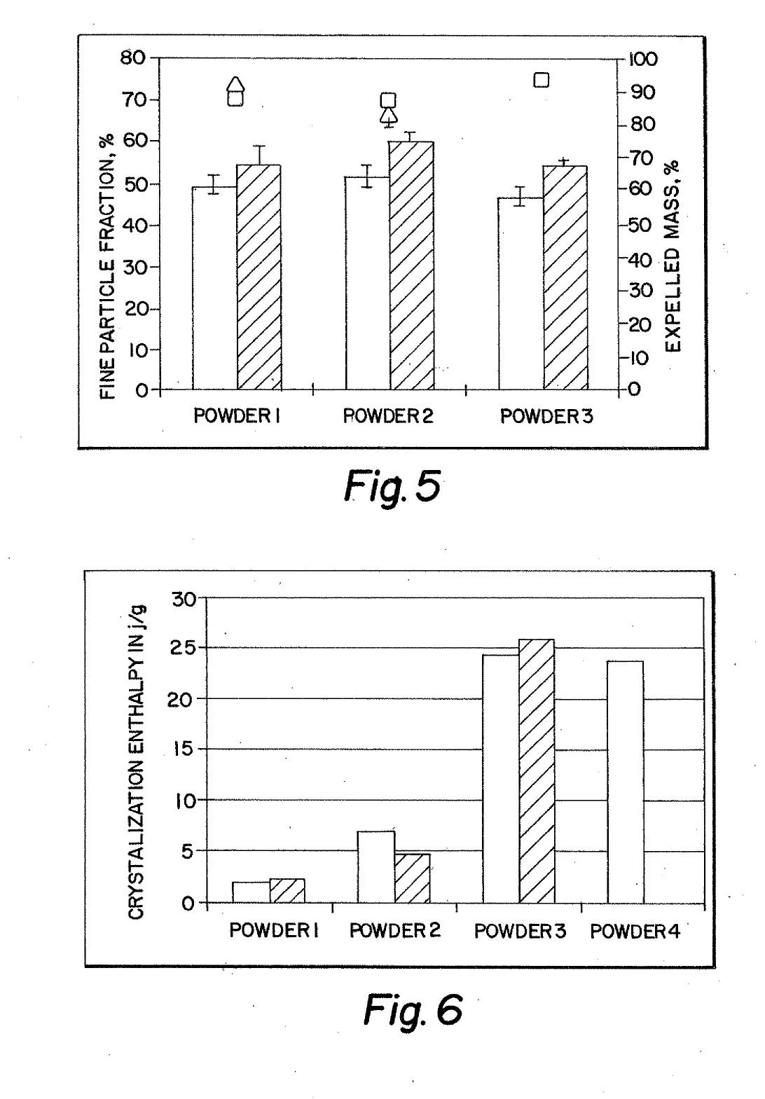

The measurement was carried out analogously to that described in the description relating to FIG. 1.

FIG. 3

Atomic force measurement (AFM) photographs of a spray-dried powder containing 80% phenylalanine, 10% LS90P and 10% IgG1 on storage at 50% RH

Preparation of sample: the powder was placed on the AFM sample disc using a spatula. An adhesive (STKY-Dot) provided the adhesive bond between the sample holder and the bottom layer of powder. The overlying layers of powder adhered by particle adhesion. Loose particles were blown away using a dry nitrogen current. Method: Directly after the preparation of the sample the powder was placed in the AFM head and the AFM-LASER was adjusted. After the adjustment the AFM was hermetically sealed using a hood (atmospheric hood) and the locked in air was dehumidified to 0% relative humidity. After the dehumidification a suitable powder particle surface was continuously scanned at one point. Once a stable scanning state had been established the humidity was increased to 50% relative humidity within a few minutes.

Materials:

AFM MultiMode™ SPM from Veeco
E-Scanner from Veeco
TIP: MPP-11200 from Veeco
Atmospheric hood from Veeco
Sample disc from Veeco
STKY-Dot from Veeco
Software Version V5.12b48
Humidity regulator UH-LFR from Boehringer Ingelheim
   Parameters:
Tapping Mode
Scan rate: 1-2 Hz
Scan resolution: 512×512 pixels
Tip frequency: 250-300 kHz
Air humidity: approx. 0% RH, 50±4% RH, 70±3% RH (Relative Humidity)
Temperature of the sample during scanning: T_{S}=22-28° C.
   a) starting value, spray-dried powder: 80% phenylalanine/10% LS90P/10% IgG1
   b) after 12 minutes incubation at 50% RH, spray-dried powder: 80% phenylalanine/10% LS90P/10% IgG1
   c) incubation period at 50% RH after 53 minutes incubation at 50% RH, spray-dried powder: 80% phenylalanine/10% LS90P/10% IgG1
   d) after 8 hours incubation at 50% RH, spray-dried powder: 80% phenylalanine/10% LS90P/10% IgG1
   e) after 20 hours incubation at 50% RH, spray-dried powder: 80% phenylalanine/10% LS90P/10% IgG1

FIG. 4

Atomic force measurement (AFM) photographs of a spray-dried powder containing 80% phenylalanine, 10% LS90P and 10% IgG1 on storage at 60% RH

The measurement was carried out analogously to that described in connection with FIG. 3.

a) starting value, spray-dried powder: 80% phenylalanine/10% LS90P/10% IgG1

b) after 12 minutes incubation at 60% RH, spray-dried powder: 80% phenylalanine/10% LS90P/10% IgG1

c) incubation period at 50% RH after 44 minutes incubation at 50% RH, spray-dried powder: 80% phenylalanine/10% LS90P/10% IgG1

d) after 8 hours incubation at 50% RH, spray-dried powder: 80% phenylalanine/10% LS90P/10% IgG1

e) after 17 hours incubation at 50% RH, spray-dried powder: 80% phenylalanine/10% LS90P/10% IgG1

FIG. 5

Comparison of the fine particle fractions of spray-dried powders before and after tempering.

The fine particle fraction was determined with a one-stage impactor (Impactor Inlet, TSI) in combination with the Aerodynamic Particle Sizer (APS, TSI). The separation threshold of the impactor nozzle was at 5.0 μm. In addition to the fine particle fraction the aerodynamic particle size was determined using the APS and the particle size distribution was determined by measuring the time of flight. To do this, the powder was split after passing through the Sample Induction Ports. A fraction of 0.2% was sucked into a small capillary under isokinetic conditions and the time of flight measuring unit was introduced. The remaining fraction was used to determine the fine particle fraction.

For measurement the powder was packed into size 3 capsules and expelled using an inhaler (HandiHaler®, Boehringer Ingelheim). The flow rate for expelling the powder was adjusted so that a pressure drop of 4 kPa prevailed through the HandiHaler. The air volume was 4 litres according to the PharmEur. To prevent “rebouncing” of the particles deposited on the impactor stage, the impactor plate has been coated with a highly viscous Brij solution for the measurements.

The expelled mass is obtained from the difference in the weight of the capsule before and after expulsion through the inhaler (HandiHaler®, Boehringer Ingelheim).

Light bar: percentage fine particle fraction before tempering

Dark bar: percentage fine particle fraction after tempering

triangles: expelled mass directly after spray drying

rectangles: expelled mass after tempering (50% RH at ambient temperature over 20 hours)

powder 1: spray-dried powder consisting of 60% phenylalanine, 30% LS90P and 10% IgG1

powder 2: spray-dried powder consisting of 60% phenylalanine, 30% LS90P and 10% lysozyme

powder 3: spray-dried powder consisting of 60% phenylalanine, 30% LS90P and 10% calcitonin

FIG. 6

DSC measurements for determining the crystallisation enthalpy of the LS90P

The crystallisation enthalpy was determined by measuring the heat currents during the heating of the powders. When an amorphous powder is heating up the constituents of the particle have increased mobility after passing through the glass transition temperature and may crystallise. Passing through the glass transition temperature is an endothermic process. The subsequent crystallisation, on the other hand, is exothermic. As the powder is heated further it may melt or decompose.

For the DSC measurements, a few milligrams of powder were slightly compressed in a crucible so as to form a bed of powder that was as homogeneous and dense as possible. Then the crucible was sealed by cold welding. The measurements were carried out with an unperforated crucible.

The other parameters were:

| | |
|---|---|
| Measuring equipment: | DSC 821/Mettler Toledo |
| Evaluating software: | STAR version 4.20 |
| furnace gas: | nitrogen/40 mL/min |
| flushing gas: | nitrogen/150 mL/min |
| crucible: | aluminium crucible, 40 μL |
| scan rate: | temperature 10° C./min |

powder 1: spray-dried powder: 60% phenylalanine/40% LS90P
powder 2: spray-dried powder: 60% phenylalanine/30% LS90P/10% IgG1
powder 3: spray-dried powder: 60% phenylalanine/30% LS90P/9% IgG1/1% HSA

powder 4: freeze-dried powder: 100% LS90P

| | |
|---|---|
| Light bar: | crystallisation enthalpy in J/g before tempering |
| Dark bar: | crystallisation enthalpy in J/g after tempering |

### DETAILED DESCRIPTION OF THE INVENTION

### Definitions

Terms and designations used within the scope of this specification have the following meanings defined below. The details of weight and percentages by weight are based on the dry mass of the compositions or the solids content of the solutions/suspensions, unless stated otherwise.

The general expressions “containing” or “contains” include the more specific term of “consisting of”. Moreover, “one” and “many” are not used restrictively.

“powders” denotes a very fine, comminuted substance. “Spray-dried powder” means a powder produced by spray drying.

“Particle” denotes a small fragment of a substance. In the present invention the term particles refers to the particles in the powders according to the invention. The terms particles and powders are occasionally used interchangeably in the present invention. The term powder also includes its constituents, the particles. Particles thus refer to all the particles, i.e. the powder.

The term “mixture” or “mixtures” in the sense of this invention refers both to those mixtures which are generated from a genuine solution of all the components or from a solution in which one or more of the components have or has been suspended. However, the term “mixtures” in the sense of this invention also refers to mixtures which have been produced by a physical mixing process from solid particles of these components or which have formed by the application of a solution or suspension of these components to one or more solid components.

The term “composition” refers to liquid, semi-solid or solid mixtures of at least two starting materials.

The term “pharmaceutical composition” refers to a composition for administering to the patient.

The term “pharmaceutically acceptable excipients” relates to excipients, which may possibly be present in the formulation within the scope of the invention. The excipients may for example be administered by pulmonary route without having any significant toxicologically harmful effects on the subjects or on the subjects' lungs.

The term “pharmaceutically acceptable salts” includes for example the following salts, but is not restricted thereto: salts of inorganic acids such as chloride, sulphate, phosphate, diphosphate, bromide and nitrate salts. Also, salts of organic acids, such as malate, maleate, fumarate, tartrate, succinate, ethylsuccinate, citrate, acetate, lactate, methanesulphonate, benzoate, ascorbate, para-toluenesulphonate, palmoate, salicylate and stearate, and also estolate, gluceptate and lactobianate salts.

By the term “active substances” are meant substances that provoke an activity or a reaction in an organism. If an active substance is administered to a human or to an animal body for therapeutic purposes, it is referred to as a pharmaceutical composition or medicament.

By a “protein active substance” is meant in the present invention an active substance which is structurally present as a protein or structurally constitutes a protein, polypeptide or peptide.

Examples of active substances are insulin, insulin-like growth factor, human growth hormone (hGH) and other growth factors, tissue plasminogen activator (tPA), erythropoietin (EPO), cytokines, e.g. interleukines (IL) such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18 interferon (IFN)-alpha, -beta, -gamma, -omega or -tau, tumour necrosis factor (TNF) such as TNF-alpha, beta or gamma, TRAIL, G-CSF, GM-CSF, M-CSF, MCP-1 and VEGF. Other examples are monoclonal, polyclonal, multispecific and single chain antibodies and fragments thereof such as for example Fab, Fab′, F(ab′)₂, Fc and Fc′ fragments, light (L) and heavy (H) immunoglobulin chains and the constant, variable or hypervariable regions thereof as well as Fv and Fd fragments (Chamov et al., 1999). The antibodies may be of human or non-human origin. Humanised and chimeric antibodies are also possible. Similarly, it relates to conjugated proteins and antibodies which are connected for example to a radioactive substance or a chemically defined medicament.

Fab fragments (fragment antigen binding=Fab) consist of the variable regions of both chains which are held together by the adjacent constant regions. They may be produced for example from conventional antibodies by treating with a protease such as papain or by DNA cloning. Other antibody fragments are F(ab′)₂ fragments which can be produced by proteolytic digestion with pepsin.

By gene cloning it is also possible to prepare shortened antibody fragments which consist only of the variable regions of the heavy (VH) and light chain (VL). These are known as Fv fragments (fragment variable=fragment of the variable part). As covalent binding via the cystein groups of the constant chains is not possible in these Fv fragments, they are often stabilised by some other method. For this purpose the variable region of the heavy and light chains are often joined together by means of a short peptide fragment of about 10 to 30 amino acids, preferably 15 amino acids. This produces a single polypeptide chain in which VH and VL are joined together by a peptide linker. Such antibody fragments are also referred to as single chain Fv fragments (scFv). Examples of scFv antibodies are known and described, cf. for example Huston et al., 1988.

In past years various strategies have been developed for producing multimeric scFv derivatives. The intention is to produce recombinant antibodies with improved pharmacokinetic properties and increased binding avidity. In order to achieve the multimerisation of the scFv fragments they are produced as fusion proteins with multimerisation domains. The multimerisation domains may be, for example, the CH3 region of an IgG or helix structures (“coiled coil structures”) such as the Leucine Zipper domains. In other strategies the interactions between the VH and VL regions of the scFv fragment are used for multimerisation (e.g. dia-, tri- and pentabodies).

The term “diabody” is used in the art to denote a bivalent homodimeric scFv derivative. Shortening the peptide linker in the scFv molecule to 5 to 10 amino acids results in the formation of homodimers by superimposing VHNL chains. The diabodies may additionally be stabilised by inserted disulphite bridges. Examples of diabodies can be found in the literature, e.g. in Perisic et al., 1994.

The term “minibody” is used in the art to denote a bivalent homodimeric scFv derivative. It consists of a fusion protein which contains the CH3 region of an immunoglobulin, preferably IgG, most preferably IgG1, as dimerisation region. This connects the scFv fragments by means of a hinge region, also of IgG, and a linker region. Examples of such minibodies are described by Hu et al., 1996.

The term “triabody” is used in the art to denote a trivalent homotrimeric scFv derivative (Kortt et al., 1997). The direct fusion of VH-VL without the use of a linker sequence leads to the formation of trimers.

The fragments known in the art as mini antibodies which have a bi-, tri- or tetravalent structure are also derivatives of scFv fragments. The multimerisation is achieved by means of di-, tri- or tetrameric coiled coil structures (Pack et al., 1993 and 1995; Lovejoy et al., 1993).

The term “excipients” refers to substances which are added to a formulation, in the present invention a powder, particularly spray-dried powder. Excipients usually have no activity themselves, particularly no pharmaceutical activity, and serve to improve the formulation of the actual ingredient, e.g. an active substance, or to optimise a particular aspect thereof (e.g. storage stability).

A pharmaceutical “excipient” is a part of a medicament or a pharmaceutical composition, and ensures among other things that the active substance reaches the activity site and is released there. Excipients have three basic tasks: a carrier function, controlling the release of active substance and increasing the stability. Excipients are also used to produce pharmaceutical forms which are thereby altered in their duration or rate of effect.

The term “amino acid” refers to compounds which contain at least one amino and at least one carboxyl group. Although the amino group is usually in the α-position to the carboxyl group, any other arrangement in the molecule is conceivable. The amino acid may also contain other functional groups, such as e.g. amino, carboxamide, carboxyl, imidazole, thio groups and other groups. Amino acids of natural or synthetic origin, racemic or optically active (D- or L-) including various stereoisomeric proportions, may be used. For example the term isoleucine includes both D-isoleucine, L-isoleucine, racemic isoleucine and various ratios of the two enantiomers.

The term “peptide”, “polypeptide” or “protein” refers to polymers of amino acids consisting of more than two amino acid groups.

Furthermore the term “peptide”, “polypeptide” or “protein” refers to polymers of amino acids consisting of more than 10 amino acid groups.

The term peptide, polypeptide or protein is used as a pseudonym and includes both homo- and heteropeptides, i.e. polymers of amino acids consisting of identical or different amino acid groups. A “di-peptide” is thus made up of two peptidically linked amino acids, a “tri-peptide” is made up of three peptidically linked amino acids.

The term “protein” used here refers to polymers of amino acids with more than 20 and particularly more than 100 amino acid groups.

The term “small protein” refers to proteins under 50 kD or under 30 kD or between 5-50 kD. The term “small protein” further relates to polymers of amino acid groups with less than 500 amino acid groups or less than 300 amino acid groups or polymers with 50-500 amino acid groups. Preferred small proteins are e.g. growth factors such as “human growth hormone/factor”, insulin, calcitonin or the like.

The term “protein stability” denotes monomer contents of more than 90%, preferably more than 95%.

The term “oligosaccharide” or “polysaccharide” refers to polysaccharides consisting of at least three monomeric sugar molecules.

The term “% (w/w)” refers to the percentage amount, based on the mass, of an active substance or an excipient in the spray-dried powder. The proportion stated is based on the dry substance of the powder. The residual moisture in the powder is thus not taken into consideration.

The term “amorphous” means that the powdered formulation contains less than 10% crystalline fractions, preferably less than 7%, more preferably less than 5%, and most preferably less than 4, 3, 2, or 1%.

The word “inhalable” means that the powders are suitable for pulmonary administration. Inhalable powders can be dispersed and inhaled by means of an inhaler so that the particles enter the lungs and are able to develop a systemic activity optionally through the alveoli. Inhalable particles may have an average particle diameter, for example, of between 0.4-30 μm (MMD=mass medium diameter), usually between 0.5-20 μm, preferably between 1-10 μm and/or an average aerodynamic particle diameter (MMAD=mass median aerodynamic diameter) of between 0.5-10 μm, preferably between 0.5-7.5 μm, more preferably between 0.5-5.5 μm, even more preferably between 1-5 μm and most preferably between 1-4.5 μm or 3-10 μm.

“Mass Median Diameter” or “MMD” is a measurement of the average particle size distribution. The results are expressed as diameters of the total volume distribution at 50% total throughflow. The MMD values can be determined for example by laser diffractometry, although of course any other conventional method may be used (e.g. electron microscopy, centrifugal sedimentation).

The term “mean aerodynamic particle diameter” (=mass median aerodynamic diameter (MMAD)) indicates the aerodynamic particle size at which 50% of the particles of the powder normally have a smaller aerodynamic diameter. In cases of doubt the reference method for determining the MMAD is the method specified in this patent specification (cf. the Chapter EXAMPLES, Method).

MMD and MMAD may differ from one another, e.g. a hollow sphere produced by spray drying may have a greater MMD than its MMAD.

The term “fine particle fraction” (FPF) describes the inhalable part of a powder consisting of particles with a particle size of ≦5 μm MMAD. In powder which is readily dispersible the FPF is more than 20%, preferably more than 30%, more particularly more than 40%, and more preferably more than 50%, even more preferably more than 55%. The expression “Cut Off Diameter” used in this context indicates which particles are taken into account when determining the FPF. An FPF of 30% with a Cut Off Diameter of 5 μm (FPF 5) means that at least 30% of all the particles in the powder have a mean aerodynamic particle diameter of less than 5 μm.

The term “relative FPF” describes the FPF in relation to an initial or starting value. For example, the relative FPF after storage is based on the FPF before storage.

The term “time of flight” is the name of a standard method of measurements, as described in more detail in the Chapter EXAMPLES. In a time of flight measurement the MMAD is determined by measuring the time of flight of a particle over a defined measured distance. The MMAD correlates with the time of flight/This means that particles with a greater MMAD take a longer time to fly than correspondingly smaller particles (cf. one this subject: Chapter EXAMPLES, Method).

The term “dispersible” means capable of flight. The basic prerequisite for the ability of a powder to fly is the disaggregation of the powder into individual particles and the distribution of the individual particles in air. Particle clumps are too big to enter the lungs and are therefore not suitable for inhalation therapy.

The term “expelled mass” states the amount of powder delivered when an inhaler is used. The delivery is determined in this case for example using a capsule, by weighing the capsule before and after the expulsion. The expelled mass corresponds to the difference in mass of the capsule before and after the expulsion.

The term “tempering” denotes carrying out a change of state. Tempering comprises the controlled exposure of an amorphous powder to humidity or to a water-containing or solvent-containing gas with a defined relative humidity at a defined temperature over an equally defined exposure period. An essential characteristic of the tempering is the controlled crystallisation of the particles by moisture. The tempering should modify the surface structure to a point where mainly crystal formation takes place on the surface. The nucleus of the particle is also amorphous. This method is further characterised in that mainly the substance which is to be crystallised is located on the surface of the particle. This is generally one or more excipients. The positive effect of the tempering is the improvement in the physicochemical properties. By limiting the crystallisation to the surface of the particle the substance or the active substance or particularly the protein is further stabilised by an amorphous environment within the nucleus of the particle. Crystallisation of the particle as a whole, however, is to be avoided. The tempering processes preferably take place at relative humidities in excess of 30%, but ideally at 50-60% relative humidity. The exposure time is dependent on the rate of crystallisation of the excipient.

The term “crystal” means a substance the smallest constituents of which such as ions, molecules and atoms are made up of crystal structures. Substances and combinations of substances are “crystalline” if “crystallinity” or “crystallisation” is detected by suitable methods. Examples of suitable analytical methods are X-ray diffraction, solution calorimetry and methods of determining hygroscopicity (for example with a DVS, Messrs Porotec). In X-ray diffraction, an X-ray beam is refracted from a crystal lattice. The crystal structure can be determined from the arrangement of the diffraction spectrum. A quantitative finding of crystallinity or crystallisation is obtained from the intensity of the reflection peaks. It is also possible to quantify crystallinity by solution calorimetry and measurement of hygroscopicity. In solution calorimetry the different shades of heat of amorphous and crystalline modifications of a solid are used for the quantifying process. The method of determining hygroscopicity makes use of the property that amorphous modification is less hygroscopic than crystalline modification.

In the analytical methods mentioned, before quantifying crystallinity a calibrating line is recorded using samples of known crystallinity.

The term “relative humidity” (RH) refers to the absorption capacity of air or nitrogen or the like for a vapour. The vapour may consist of water or some other organic solvent. By the relative humidity is meant the ratio of the actual mass of vapour obtained in the air or nitrogen or the like to the maximum possible mass.

The term “vapour” means the gaseous aggregate state of a substance into which the substance goes as a result of boiling or sublimation. The vapour may consist of both water and an organic solvent. Of the organic solvents, pharmaceutically acceptable substances are preferred, such as for example ethanol or isopropanol. Furthermore in special cases the following organic solvents may be used, such as glucofurol, ethyl lactate, N-methyl-2-pyrollidone, dimethyl sulphoxide, ethyleneglycol or low-chained saturated hydrocarbons such as for example pentane, hexane, heptane. However, the application is not restricted to these examples.

The terms “vapour” and “gas”, “water-containing gas” and “water-containing vapour” or “solvent-containing gas” and “solvent-containing vapour” are used interchangeably. The meaning of these terms will be apparent from the definition for vapour.

The term “ambient temperature” denotes a temperature of approx. 20-25° C. (+/−10%). The term ambient temperature denotes in particular a temperature of 25° C.

The term “monomer content” and “monomer” denotes the percentage proportion of protein consisting of a single subunit of the protein. A distinction must be drawn between the monomer content and fractions consisting of small fragments of the monomer and di- or oligomers consisting of several subunits. The monomer content mentioned in the patent specification is determined by exclusion chromatography.

The term “aggregates” refers to the proportion of di- and oligomers of proteins that consist of a single subunit in the native state.

### COMPOSITIONS ACCORDING TO THE INVENTION

The present invention relates to the modification of surfaces in powders, particularly spray-dried powders, by a controlled exposure of the powders to humidity/temperature. This produces crystals on the surface. This process is hereinafter referred to as tempering.

The crux of the invention is directed to optimising the flowability and improving the aerodynamic and electrostatic properties of the powders.

The present invention relates to a method of increasing, maintaining or minimising the reduction in the flowability (FPF) of a powder containing an active substance, particularly a protein, and at least one excipient, characterised in that
an amorphous powder is exposed
over a defined exposure period
in controlled manner to a water-containing gas or a solvent-containing gas with a defined relative humidity at a defined temperature.

The present invention preferably relates to a method according to the invention wherein the exposure period is selected such that the excipient crystallises before the active substance.

In this method according to the invention it is preferable to use crystallisation inhibitors such as HSA (human serum albumin). Preferably the powder contains at least 0.1% (w/w) HSA, at least 0.5% (w/w) HSA, at least 1% (w/w) HSA, at least 5% (w/w) HSA, at least 10% (w/w) HSA, at least 15% (w/w) HSA. Furthermore the powder preferably contains between 0.1% (w/w)-60% (w/w) HSA, 0.5% (w/w)-60% (w/w) HSA, 1% (w/w)-60% (w/w) HSA, 10% (w/w)-60% (w/w) HSA, 0.1% (w/w)-40% (w/w) HSA, 0.5% (w/w)-40% (w/w) HSA, 1% (w/w)-40% (w/w) HSA, 10% (w/w)-40% (w/w) HSA, 0.1% (w/w)-20% (w/w) HSA, 0.5% (w/w)-20% (w/w) HSA, 1% (w/w)-20% (w/w) HSA, 10% (w/w)-20% (w/w) HSA, 0.1% (w/w)-1% (w/w) HSA, 0.5% (w/w)-1% (w/w) HSA, 0.1% (w/w)-0.90% (w/w) HSA, 0.5% (w/w)-0.9% (w/w) HSA, 0.1% (w/w)-3% (w/w) HSA, 0.5% (w/w)-3% (w/w) HSA. Furthermore the powder preferably contains less than 1% (w/w) HSA, less than 0.9% (w/w) HSA.

The present invention preferably further relates to a method according to the invention wherein the relative humidity of the water-containing or solvent-containing gas is greater than 30% (w/w), preferably between 50-60% (w/w).

In a particularly preferred embodiment the excipient is phenylalanine.

The present invention preferably further relates to a method according to the invention wherein the amount of excipient is at least 10% (w/w). A preferred excipient is phenylalanine. A particularly preferred embodiment is therefore a method according to the invention wherein at least 10% (w/w) phenylalanine are used as excipient. Furthermore phenylalanine contents of at least 30% (w/w) and at least 40% (w/w) are also preferred.

In a preferred embodiment the process according to the invention is carried out while retaining the stability of the substance. The stability of the substance is retained or improved, particularly the storage stability and particularly under raised humidity conditions.

In a special embodiment of the method according to the invention the FPF of the powder after three months' storage at humidities of 60% (w/w) relative humidity after the process (in which case it is a relative FPF=rFPF, i.e. based on the starting value) of more than 60%, 70%, 80%, 90%, 95% of the starting value (before the process).

In another special embodiment of the method according to the invention the stability of the substance is maintained or improved, particularly the storage stability and particularly at raised relative humidity.

Storage is over 3 months or 6 months, for example.

In a preferred embodiment of the method according to the invention the temperature is less than 60° C.

In a preferred embodiment the powder in question is a spray-dried powder.

In a special embodiment the invention relates to powders containing a protein or a protein-active substance and phenylalanine as excipient and optionally a sugar, while the powder is characterised in that it contains at least 10% (w/w), at least 30%, at least 40% (w/w) phenylalanine, preferably 10% (w/w) and particularly preferably 30% (w/w). Optionally other substances particularly other excipients may be contained in the powder. Furthermore this special embodiment of the present invention also relates to a pharmaceutical composition which contains a powder, consisting of a protein or a protein-active substance and phenylalanine as excipient and optionally a sugar, while the powder consists of at least 10% (w/w), at least 30%, at least 40% (w/w) phenylalanine, preferably 10% (w/w) and particularly preferably 30% (w/w).

A preferred embodiment of the method according to the invention relates to a method of increasing the FPF, in particular by at least 6%, preferably 7%, 8%, 9%, 10%, 11%, 12%, 13%, 14% or more than 14%.

The invention further relates to a method of improving the aerodynamic properties of a powder containing an active substance, particularly a protein, and at least one excipient, characterised in that
an amorphous powder is exposed
over a defined exposure period
in controlled manner to a water-containing gas or a solvent-containing gas with a defined relative humidity at a defined temperature.

The stability of the powder is preferably maintained.

The present invention preferably relates to a method according to the invention of improving the aerodynamic properties of a powder in which the exposure period is selected such that the excipient crystallises before the active substance.

In this method according to the invention it is also preferable to use crystallisation inhibitors such as HSA. Preferably the powder contains at least 0.1% (w/w) HSA, at least 0.5% (w/w) HSA, at least 1% (w/w) HSA, at least 5% (w/w) HSA, at least 10% (w/w) HSA, at least 15% (w/w) HSA. Furthermore the powder preferably contains between 0.1% (w/w)-60% (w/w) HSA, 0.5% (w/w)-60% (w/w) HSA, 1% (w/w)-60% (w/w) HSA, 10% (w/w)-60% (w/w) HSA, 0.1% (w/w)-40% (w/w) HSA, 0.5% (w/w)-40% (w/w) HSA, 1% (w/w)-40% (w/w) HSA, 10% (w/w)-40% (w/w) HSA, 0.1% (w/w)-20% (w/w) HSA, 0.5% (w/w)-20% (w/w) HSA, 1% (w/w)-20% (w/w) HSA, 10% (w/w)-20% (w/w) HSA, 0.1% (w/w)-1% (w/w) HSA, 0.5% (w/w)-1% (w/w) HSA, 0.1% (w/w)-0.90% (w/w) HSA, 0.5% (w/w)-0.9% (w/w) HSA, 0.1% (w/w)-3% (w/w) HSA, 0.5% (w/w)-3% (w/w) HSA. Furthermore the powder preferably contains less than 1% (w/w) HSA, less than 0.9% (w/w) HSA.

The present invention preferably further relates to a method according to the invention of improving the aerodynamic properties of a powder in which the relative humidity of the water-containing or solvent-containing gas is more than 30% (w/w), preferably between 50-60% (w/w).

The temperature is preferably below 60° C.

The invention further relates to a method of reducing the electrostatics of a powder containing an active substance, particularly a protein, and at least one excipient. characterised in that
an amorphous powder is exposed
over a defined exposure period
in controlled manner to a water-containing gas or a solvent-containing gas with a defined relative humidity at a defined temperature.

The present invention preferably relates to a method according to the invention of reducing the electrostatics of a powder in which the exposure period is selected such that the excipient crystallises before the active substance.

The present invention preferably further relates to a method according to the invention of reducing the electrostatics of a powder in which the relative humidity of the water-containing or solvent-containing gas is greater than 30% (w/w), preferably between 50-60% (w/w).

The temperature is preferably below 60° C.

In a preferred embodiment of the method of reducing the electrostatics of a powder the invention relates to powders containing a crystallisation inhibitor such as HSA. Preferably the powder contains at least 0.1% (w/w) HSA, at least 0.5% (w/w) HSA, at least 1% (w/w) HSA, at least 5% (w/w) HSA, at least 10% (w/w) HSA, at least 15% (w/w) HSA. Furthermore the powder preferably contains between 0.1% (w/w)-60% (w/w) HSA, 0.5% (w/w)-60% (w/w) HSA, 1% (w/w)-60% (w/w) HSA, 10% (w/w)-60% (w/w) HSA, 0.1% (w/w)-40% (w/w) HSA, 0.5% (w/w)-40% (w/w) HSA, 1% (w/w)-40% (w/w) HSA, 10% (w/w)-40% (w/w) HSA, 0.1% (w/w)-20% (w/w) HSA, 0.5% (w/w)-20% (w/w) HSA, 1% (w/w)-20% (w/w) HSA, 10% (w/w)-20% (w/w) HSA, 0.1% (w/w)-1% (w/w) HSA, 0.5% (w/w)-1% (w/w) HSA, 0.1% (w/w)-0.90% (w/w) HSA, 0.5% (w/w)-0.9% (w/w) HSA, 0.1% (w/w)-3% (w/w) HSA, 0.5% (w/w)-3% (w/w) HSA. Furthermore the powder preferably contains less than 1% (w/w) HSA, less than 0.9% (w/w) HSA.

In a special embodiment the invention relates to a method of filling powders, characterised in that the powders have been treated according to the method described.

The present method relates to volumetric and mass-dependent filling, e.g. with a pipette, a filling roller or a gravity dispenser. The improved fillability thanks to an additional tempering step is characterised in that as a result of the consequent improvement in flowability and reduction in the electrostatic charging of the powders the filling times are reduced and the filling precision is improved.

In one embodiment of the method according to the invention the exposure time is at least 8 hours or more, at least 12 hours or more, at least 20 hours or more, preferably 20 hours and particularly preferably 20 hours.

In a further embodiment of the method according to the invention the temperature during the exposure time is less than 60° C., particularly between −10° C. to 60° C., preferably 4° C. to 40° C. and particularly preferably between 16° C. and 35° C.

In a further preferred embodiment of the method according to the invention the temperature during the exposure time is 4° C., 10° C., ambient temperature or 37° C., preferably ambient temperature.

In a preferred embodiment the active substance in the method according to the invention is a protein such as for example insulin, insulin-like growth factor, human growth hormone (hGH) and other growth factors, tissue plasminogen activator (tPA), erythropoietin (EPO), cytokines, e.g. interleukines (IL) such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18 interferon (IFN)-alpha, -beta, -gamma, -omega or -tau, tumour necrosis factor (TNF) such as TNF-alpha, beta or gamma, TRAIL, G-CSF, GM-CSF, M-CSF, MCP-1 and VEGF. Other examples are monoclonal, polyclonal, multispecific and single chain antibodies and fragments thereof such as for example Fab, Fab′, F(ab′)₂, Fc and Fc′ fragments, light (L) and heavy (H) immunoglobulin chains and the constant, variable or hypervariable regions thereof as well as Fv and Fd fragments (Chamov et al., 1999). The antibodies may be of human or non-human origin. Humanised and chimeric antibodies are also possible.

The invention further relates to powders with raised, maintained or minimally reduced flowability (FPF) or improved aerodynamic or electrostatic properties which can be prepared by the methods according to the invention.

The invention particularly relates to powders with increased flowability or increased nano-roughness, which can be obtained by one of the methods described according to the invention.

In a further embodiment of the present method for increasing, maintaining or minimising the reduction in the flowability (FPF) of a powder or for improving the aerodynamic or electrostatic properties of a powder, the powder contains a substance 1 and at least one other substance 2, wherein substance 2 crystallises before substance 1.

The present invention thus further relates to a method of increasing, maintaining or minimising the reduction in the flowability (FPF) of a powder containing a substance 1, particularly a protein, and at least one substance 2, characterised in that
an amorphous powder is exposed
over a defined exposure period
in controlled manner to a water-containing gas or a solvent-containing gas with a defined relative humidity at a defined temperature,
wherein substance 2 crystallises before substance 1.

A preferred embodiment of the present invention relates to methods which exclude further coating with further particles, e.g. which exclude coating with Mg-stearate or phospholipids.

Another preferred embodiment of the present invention relates to methods that exclude mixing with particles such as tiny leucine particles or generally with nanoscale particles, but also with substantially larger carriers. A particular embodiment of the method thus relates to methods which exclude mixing with other particles.

A preferred embodiment of the present invention relates to a method which conditions amorphous or partly crystalline powders without the use of supercritical or subcritical media. In a preferred embodiment the present invention thus excludes supercritical methods or the use of supercritical or subcritical media.

The present invention thus further relates to a method for increasing, maintaining or minimising the reduction in the flowability (FPF) of a powder or for improving the aerodynamic or electrostatic properties of a powder containing an active substance, particularly a protein, and at least one excipient, characterised in that
an amorphous powder is exposed
over a defined exposure period
in controlled manner to a water-containing gas or a solvent-containing gas with a defined relative humidity at a defined temperature,
wherein the application or use of supercritical or subcritical media is excluded.

It is apparent from the following experiments that the optimising of the aerodynamic characteristics by tempering is not restricted to antibodies but is also possible in other categories of proteins such as for example enzymes (e.g. lysozyme) and hormones (e.g. calcitonin).

It is also apparent from the following experiments that the novel powder is optimised with respect to its aerodynamic characteristics or flowability by controlled exposure to humidity, while retaining the protein stability. The optimising of the powder properties is accompanied by surface crystallisation of the particle surface.

Particular emphasis should be placed on the addition of hydrophobic or poorly soluble substances to the spray solution, while after drying this substance can crystallise well and in controllable manner under the effects of humidity. Thus, for example, phenylalanine exhibits this characteristic, particularly with a phenylalanine content in the powder of at least 10% (w/w), at least 30% (w/w) or at least 40% (w/w), at least 10% (w/w) being preferred. This amino acid accumulates on the droplet surface because of the hydrophobicity in the spray droplets. As a result of the lower solubility compared with antibodies and the sugars or polyols normally used, such as e.g. saccharose or mannitol, when the droplet is evaporated first a solid layer is formed consisting mainly of phenylalanine. Because of the hydrophobicity and the poor solubility the phenylalanine accumulates on the particle surface in the dried particles. There is an at least partial separation between a phenylalanine-rich phase on the particle surface and a phenylalanine-poor phase in the nucleus of the particle. On the other hand, the active substance and optionally other readily soluble excipients accumulate In the nucleus of the particle.

As a result of the tendency of the phenylalanine to crystallise easily, the surface of the particle may be crystallised in controlled manner thanks to the layered structure of the particle without damaging the protein in the nucleus.

The fundamental prerequisite for the tempering is a layered structure of the powders. This means that the powder components used are not homogeneously distributed in the particle but may accumulate in specific areas or layers of the particle depending on the physicochemical properties of the components. For tempering the particle it is preferable that the crystallisable components should accumulate on the outer layers of the particle.

It has been demonstrated experimentally (Example 5) that two endothermic effects can be detected. These endothermic effects correspond to two glass transition temperatures and indicate that the substances used are not homogeneously distributed in the particle. If they were homogeneously distributed in the powder particle only one glass transition temperature would be detectable which could be calculated using the Gordon-Taylor equation (L Mackin, International Journal of Pharmaceutics 231 (2002) 227-236).

Several studies have shown that the surface composition of the spray droplet correlates with the surface composition in the spray-dried powder (Fäldt et al. 1994, The surface composition of spray dried protein-lactose powders. Colloid Surf A 90, 183-190/Elversson, J. et al., In situ coating—an approach for particle modification and encapsulation of proteins during spray drying, Int. J. Pharm (2006), 323, 52-63). Therefore the surface activity of the individual components was determined by tensiometry in the solution according to the invention. It was thus possible to show experimentally (Example 5) that LS90P does not have a higher surface activity than water, so that the sugar does not accumulate on the surface after the atomising of the spray solution. A spray solution with a composition in the powder made up of 60% phenylalanine/30% LS90P/10% IgG1 showed the lowest surface tension. The reduction in the surface tension can be put down to the addition of the phenylalanine. According to these results the phenylalanine accumulates on the surface of the droplets. Combined with DSC data of the same spray-dried powder a powder is obtained as a result of phase separation of the two excipients LS90P and phenylalanine occurring during spray drying and the phenylalanine forming the outer layer in the particle and accordingly the LS90P forming the inner layer in the particle.

The following Examples are provided to illustrate the present invention, and should not be construed as limiting thereof. All references cited herein are incorporated by reference in the application in their entireties.

### EXAMPLES

### Example 1 Humidity-Induced Crystallisation of Surfaces (Tempering)

A Spray solution was prepared consisting of phenylalanine, LS90P and IgG1 in the ratio 80/10/10. The solid fraction of the spray solution was 3.83% (w/v).

The solution was dried under following conditions:

| | |
|---|---|
| spray dryer: | SD-Micro (Messrs. Niro) |
| entry temperature | 120° C. |
| exit temperature: | 90° C. |
| atomiser gas rate: | 4 kg/h |
| drying gas rate: | 28 kg/h |

The spray-dried powders was exposed to different humidities in the DVS. During measurement the water vapour sorption/desorption was determined as a function of the relative humidity. It is found that the present powders undergoes a loss of mass at a critical humidity of 50% (FIG. 1). This loss of mass is accompanied by recrystallisation of the powder. It is also apparent that the loss of mass is very slight, indicating that the powder has only partially crystallised.

The kinetics and extent of the crystallisation are also dependent on the humidity. It was found that at 50% RH the speed of crystallisation is substantially slower than at 60% RH (FIGS. 2a, 2b). At 60% RH moreover the residual moisture of the powder after crystallisation is significantly less than after crystallisation at 50% RH. This indicates that at 60% RH the degree of crystallisation is higher.

Morphological Investigations

Under the atomic force microscope the powder was exposed to humidity under controlled conditions and morphological changes as a function of the exposure time to humidity were determined.

For this, the powder was first dried down and then exposed to the target humidity. The powder was scanned at regular intervals. The target humidities were 50% RH and 60% RH.

The AFM photographs (FIGS. 3 and 4) show that crystallisation can be induced in the particles depending on the humidity and as a result the surface roughness increases. It also became apparent that the powder absorbs water very rapidly. At 50% or 60% the powder has absorbed enough water within about 1 hour for recrystallisation effects to set in.

### Example 2 Effect of the Tempering on the Aerodynamics and Protein Stabilisation

In this Example various spray-dried powders consisting of phenylalanine, LS90P and IgG1 were prepared. (cf. Table 1 and 2).

TABLE 1
Composition of spray solution

| | solution 1 (w/v) | solution 2 (w/v) | solution 3 (w/v) |
|---|---|---|---|
| phenylalanine: | 2.29 g/100 mL | 3.06 g/100 mL | 2.29 g/100 mL |
| IgG1: | 1.15 g/100 mL | 338 g/100 mL | 383 mg/100 mL |
| LS90P: | 383 mg/100 mL | 383 mg/100 mL | 1.15 g/100 mL |
| solid fraction: | 3.82% | 3.82% | 3.82% |
| ratio of | 3:1 | 1:1 | 1:3 |
| protein/sugar | | | |

The phenylalanine was dissolved with heating (80° C.). After the solution had cooled to ambient temperature the protein and sugar were added.

The solutions were spray-dried under the following spray conditions:

| | |
|---|---|
| spray dryer: | SD-Micro (Messrs. Niro) |
| entry temperature | 150° C. |
| exit temperature: | 90° C. |
| atomiser gas rate: | 4 kg/h |
| drying gas rate: | 28 kg/h |

TABLE 2
Composition of spray-dried powders (based on the dry substance)

| | powder 1 | powder 2 | powder 3 |
|---|---|---|---|
| phenylalanine: | 60% w/w | 80% w/w | 60% w/w |
| IgG1: | 30% w/w | 10% w/w | 10% w/w |
| LS90P: | 10% w/w | 10% w/w | 30% w/w |
| ratio of | 3:1 | 1:1 | 1:3 |
| protein/sugar | | | |

The prepared powders were tempered at 50% relative humidity over 20 hours.

TABLE 3
Aerodynamic properties without tempering
powders after spray drying without tempering, (Ph/LS90P/IgG1)

| | 60/10/30 | 80/10/10 | 60/30/10 |
|---|---|---|---|
| MMAD [μm] | 4.25 | 3.77 | 3.73 |
| FPF [%] | 59.63 | 51.20 | 42.78 |
| EM [%] | 89.93 | 91.83 | 73.27 |
| Mon. [%] | 97.00 | 92.00 | 96.30 |
| Aggr.[%] | 2.70 | 7.60 | 3.30 |

TABLE 4
Aerodynamic properties with tempering
tempered, spray-dried powder (50% RH/20 Std), (Ph/LS90P/IgG1)

| | 60/10/30 | 80/10/10 | 60/30/10 |
|---|---|---|---|
| MMAD [μm] | 4.03 | 3.40 | 3.56 |
| FPF [%] | 65.13 | 58.57 | 56.73 |
| EM [%] | 95.27 | 90.23 | 93.20 |
| Mon. [%] | 97.10 | 89.80 | 96.00 |
| Aggr.[%] | 2.50 | 9.60 | 3.50 |

The tempering process improved the aerodynamic characteristics in the powders tested. The fine particle fraction in particle increased as a result of the tempering. The protein was stabilised by the tempering process, so that there was no humidity induced damage. As can be seen from the above Table, the monomer content is almost unchanged after tempering.

The improvement in the aerodynamics with phenylalanine can presumably be put down to 2 effects. As shown in Example 1, small crystals form on the particle surface in the phenylalanine-containing powder as a result of the effects of humidity. These act on the one hand as spacers. On the other hand, the crystalline surfaces are far less hygroscopic, so that less capillary forces occur as a result of steam condensation.

### Example 3 Tempering Effects Depending on the Amount of Excipient

This Example is intended to show how the tempering effect behaves as a function of the amount of excipient which is to be crystallised. For this, phenylalanine was used as the crystallisable component and its proportion was reduced from 50% to 5% in the spray-dried powder. The compositions of the powders are shown in Table 5 and the spray conditions in Table 6.

TABLE 5
Composition of powders in percent by mass

| | IgG1 | LS90P | phenylalanine |
|---|---|---|---|
| powder 1 | 30 | 20 | 50 |
| powder 2 | 30 | 30 | 40 |
| powder 3 | 30 | 40 | 30 |
| powder 4 | 30 | 50 | 20 |
| powder 5 | 30 | 60 | 10 |
| powder 6 | 30 | 65 | 5 |

TABLE 6
Spray conditions

| | |
|---|---|
| spray dryer | Büchi B191 |
| solid fraction | 3.8% w/v |
| entry temperature | 150° C. |
| exit temperature | 90° C. |
| atomiser gas rate | 700 L/h |
| drying gas rate | 100% aspirator power |

After spray drying the powders were tempered over 20 hours at 50% relative humidity and ambient temperature.

Table 7 shows the monomer contents of the powders before and after tempering. It is found that the tempering does not cause any damage to the IgG 1-antibody, since after tempering the monomer contents do not become significantly lower.

TABLE 7

| | monomer | monomer |
|---|---|---|
| | content %, | content %, |
| | before | after |
| | tempering | tempering |
| powder 1 | 98.02 | 98.30 |
| powder 2 | 98.60 | 98.62 |
| powder 3 | 98.83 | 98.83 |
| powder 4 | 98.84 | 98.79 |
| powder 5 | 98.85 | 99.20 |
| powder 6 | 99.02 | 99.23 |

By tempering the powder the aerodynamic characteristics could be improved up to phenylalanine contents of 10% (cf. Table 8). Both the fine particle fraction and the expelled mass could be increased by tempering in powders 1-5. At a 5% phenylalanine content both the fine particle fraction and the expelled mass fall. Thus, the tempering effect may not occur if the proportions of crystallisable substances are too low.

TABLE 8

| | | percentage rise |
|---|---|---|
| | percentage rise in the | in the expelled |
| | FPF after tempering | mass after tempering |
| powder 1 | 31 | 2 |
| powder 2 | 23 | 5 |
| powder 3 | 22 | 3 |
| powder 4 | 12 | 25 |
| powders 5 | 50 | 38 |
| powders 6 | −54 | −19 |

### Example 4 Tempering Effect as a Function of the Protein Used

In this Example different proteins were spray-dried with the excipients LS90P and phenylalanine and then tempered. The intention is to shown that the tempering effect for optimising the powder qualities is not restricted to one category of proteins but that the tempering may be used regardless of the protein. The compositions of the powders are listed in Table 9 and the spray conditions in Table 10.

TABLE 9
Composition of powders in percent by mass

| | | | |
|---|---|---|---|
| powder 1 | 30% IgG1 | 30% LS90P | 60% phenylalanine |
| powder 2 | 30% lysozyme | 30% LS90P | 60% phenylalanine |
| powder 3 | 30% calcitonin | 30% LS90P | 60% phenylalanine |

TABLE 10
Spray conditions

| | |
|---|---|
| spray dryer | Büchi B191 |
| solid fraction | 3.8% w/v |
| entry temperature | 150° C. |
| exit temperature | 90° C. |
| atomiser gas rate | 700 L/h |
| drying gas rate | 100% aspirator power |

FIG. 5 shows the fine particle fraction and the expelled masses of the prepared powders before and after tempering. According to this, the fine particle fraction could be improved by tempering the powders. The fine particle fractions of the prepared powders 1-3 are similarly high both before and after tempering. The expelled masses show no major differences as a function of the protein used. This means that the optimising of the aerodynamic characteristics by tempering is not restricted to antibodies of the IgG1 type, but is also possible, as shown in this Example, in enzymes (e.g. lysozyme) and hormones (e.g. calcitonin).

### Example 5 Investigations into the Layered Model of Spray-Dried Powders

The fundamental prerequisite for the tempering is a layered structure of the powders. This means that the powder components used are not homogeneously distributed in the particle but may accumulate in specific areas or layers of the particle depending on the physicochemical properties of the components. For tempering the particle it is preferable that the crystallisable components should accumulate on the outer layers of the particle.

This Example is intended to examine whether layer formation in the particles or phase separation of the excipients has taken place. For this, the glass transition temperatures were determined by calorimetry (DSC) using a spray-dried powder consisting of 60% phenylalanine, 30% LS90P and 10% IgG1. The spray conditions are given in Table 11 and the parameters of the DSC method in Table 12. The DSC measurements were carried out using an unperforated crucible. The results are based on the average of 6 individual measurements. The onset and median of the glass transition temperature were evaluated.

When the powder was heated up, 2 endothermic effects could be detected:

| | |
|---|---|
| Effect 1: | Onset: 38.3° C./median: 41.7° C. |
| Effect 2: | Onset: 127.6° C./median: 131.7° C. |

These endothermic effects correspond to two glass transition temperatures and indicate that the substances used are not homogeneously distributed in the particle. If they were homogeneously distributed in the powder particle only one glass transition temperature would be detectable which could be calculated using the Gordon-Taylor equation (L Mackin, International Journal of Pharmaceutics 231 (2002) 227-236).

TABLE 11

| | |
|---|---|
| spray dryer | SDMico, Niro |
| solid fraction | 3.8% w/v |
| entry temperature | 150° C. |
| exit temperature | 95° C. |
| atomiser gas rate | 4 kg/h |
| drying gas rate | 28 kg/h |

TABLE 12

| | |
|---|---|
| Measuring | DSC 821/Mettler Toledo |
| equipment | |
| Evaluating software | STAR version 4.20 |
| furnace gas | nitrogen/40 mL/min |
| flushing gas | nitrogen/150 mL/min |
| crucible | aluminium crucible, 40 μL |
| | cold-welded |
| weight of powder | 1.8 mg-6.5 mg |
| scan rate | 10° K/min |
| temperature | |

Furthermore the surface activity of the individual components were determined by tensiometry in the solution. Several studies have shown that the surface composition of the spray droplet correlates with the surface composition in the spray-dried powder (Fäldt et al. 1994, The surface composition of spray dried protein-lactose powders. Colloid Surf A 90, 183-190/Elversson, J. et al., In situ coating—an approach for particle modification and encapsulation of proteins during spray drying, Int. J. Pharm (2006), 323, 52-63).

Table 13 lists the spray solutions tested. Solution 4 corresponds to a spray solution typical of this patent specification with a composition in the powder of 60% phenylalanine/30% LS90P/10% IgG1.

TABLE 13
Compositions of the solutions

| | solution 1 | solution 2 | solution 3 | solution 4 |
|---|---|---|---|---|
| LS90P, | purified water | 1.143 | 1.143 | 1.143 |
| g/100 mL | | | | |
| phenylalanine, | | — | — | 2.286 |
| g/100 mL | | | | |
| IgG1, | | — | 0.381 | 0.381 |
| g/100 mL | | | | |

The surface tensions obtained were:

solution 1: 72 mN/m

solution 2: 72 mN/m

solution 3: 65 mN/m

solution 4: 59 mN/m

LS90P does not have a higher surface activity than water, so that the sugar does not accumulate on the surface after the atomising of the spray solution. The spray solution 4 shows the lowest surface tension. The reduction in the surface tension can be put down to the addition of the phenylalanine. According to these results the phenylalanine accumulates on the surface of the droplets. Combined with the DSC data of the spray-dried powder mentioned in this example a powder is obtained as a result of phase separation of the two excipients LS90P and phenylalanine occurring during spray drying and the phenylalanine forming the outer layer in the particle and accordingly the LS90P forming the inner layer in the particle.

### Example 6 Spray Drying Using Crystallisation Inhibitors

This Example is intended to show that by using crystallisation inhibitors the spray-dried powders may be further optimised. For this purpose, various powders were prepared as shown in Table 14.

TABLE 14
Compositions of the powders

| | composition | method of preparation |
|---|---|---|
| powder 1 | 60% phenylalanine | spray drying (SDMicro) |
| | 40% LS90P | |
| powder 2 | 60% phenylalanine | spray drying (SDMicro) |
| | 30% LS90P | |
| | 10% IgG1 | |
| powder 3 | 60% phenylalanine | spray drying (SDMicro) |
| | 30% LS90P | |
| | 1% HSA | |
| | 9% IgG1 | |
| powder 4 | 100% LS90P | freeze-drying (GT-12B) |

The spray conditions on the SDMicro are compiled in Table 15.

TABLE 15
Spray conditions

| | |
|---|---|
| spray dryer | SDMicro |
| solid fraction | 3.8% |
| entry temperature | 150° C. |
| exit temperature | 90° C. |
| atomiser gas rate | 4 kg/h |
| drying gas rate | 28 kg/h |

The purpose of freeze-drying an aqueous LS90P solution was to prepare X-ray-amorphous powder. For this, an aqueous solution with a small solid fraction (5 g/100 mL) was prepared and freeze-dried as described in Table 16.

TABLE 16
Temperature and pressure programme of the freeze-drying

| | time | temperature | pressure |
|---|---|---|---|
| process step | [hh:mm] | [° C.] | [mbar] |
| Start | — | 20 | — |
| freezing (temperature | 01:30 | −50 | — |
| gradient) | | | |
| freezing (holding step) | 06:30 | −50 | — |
| after-drying | 00:01 | −50 | 0.016 |
| (pressure gradient) | | | |
| main drying | 07:00 | −40 | 0.016 |
| (temperature gradient) | | | |
| main drying | 23:00 | −40 | 0.016 |
| (holding step) | | | |
| main drying | 03:20 | −23 | 0.016 |
| (temperature gradient) | | | |
| main drying | 30:00 | −23 | 0.016 |
| (holding step) | | | |
| main drying | 02:00 | 20 | 0.016 |
| (temperature gradient) | | | |
| after-drying | 00:01 | 20 | 0.001 |
| (pressure gradient) | | | |
| after-drying | 17:00 | 20 | 0.001 |
| (holding step) | | | |

FIG. 6 shows the recrystallisation enthalpies of LS90P after heating the powders in a DSC apparatus (DSC821/Mettler Toledo). It is found that the crystallisation enthalpy is greatly increased based on the proportion by mass as a result of the addition of 1% HSA. Thus, the crystallisation enthalpy of the LS90P increases before tempering from 6.80 J/g to 24.3 J/g and after tempering from 4.8 J/g to 26.0 J/g. This means that the addition of 1% HSA increases the amorphicity of LS90P.

### Example 7 Spray Drying of Other Powders Containing IgG1/LS90P and a Further Excipient

In this Example other excipients are investigated for their tempering properties.

For this, 2 powders were prepared according to Table 17 with the spray conditions according to Table 18.

TABLE 17
Composition of powders in percent by mass

| | | | |
|---|---|---|---|
| powder 1 | 30% IgG1 | 30% LS90P | 60% valine |
| powder 2 | 30% IgG1 | 30% LS90P | 60% glutamine |

TABLE 18
Spray conditions

| | |
|---|---|
| spray dryer | Büchi B191 |
| solid fraction | 3.8% w/v |
| entry temperature | 150° C. |
| exit temperature | 90° C. |
| atomiser gas rate | 700 L/h |
| drying gas rate | 100% aspirator power |

As shown in Table 19, the FPF can be improved by tempering.

Besides the improvement in the aerodynamic characteristics obtained by tempering, the protein integrity (monomer content) can also be improved by tempering as described in this Example (cf. Table 20). The monomer content is significantly higher after tempering, particularly in the case of powder 1.

TABLE 19

| | | | expelled mass | expelled |
|---|---|---|---|---|
| | FPF in % | FPF in %, | in %, | mass in %, |
| powders | untempered | tempered | untempered | tempered |
| powder 1 | 32.6 | 38.6 | 64.4 | 68.6 |
| powder 2 | 10.2 | 19.4 | 84.1 | 80.9 |

TABLE 20

| | monomer | monomer |
|---|---|---|
| | content in % | content in % |
| powders | untempered | tempered |
| powder 1 | 84.4 | 92.6 |
| powder 2 | 98.3 | 98.9 |

1. A method of increasing, maintaining or minimising the reduction in the flowability (FPF) of a powder comprising the step of exposing an amorphous powder comprising an active substance and at least one excipient (i) over a defined exposure period (ii) in a controlled manner to a water-containing gas or a solvent-containing gas with a defined relative humidity at a defined temperature.

2. The method according to claim 1, wherein said active substance is a protein.

3. The method according to claim 1, wherein said exposure period is selected such that the excipient crystallises before the active substance.

4. The method according to claim 1, wherein the relative humidity of the water-containing or solvent-containing gas is greater than 30% (w/w) or between 50-60% (w/w).

5. The method according to claim 1, wherein the relative FPF of the powder after three months' storage at humidities of 60% (w/w) relative humidity after the process is more than 60%, 70%, 80%, 90%, or 95% of the starting value.

6. The method according to claim 1, wherein the stability of said substance is maintained or improved when compared to a substance not subjected to said step b).

7. The method according to claim 6, wherein the storage stability of said substance is maintained or improved.

8. The method according to claim 6, wherein the storage stability of said substance is maintained or improved when said substance is stored at elevated relative humidity.

9. The method according to claim 1, wherein the FPF of said powder is increased by at least 6%, by at least 7%, by at least 8%, by at least 9%, by at least 10%, by at least 11%, by at least 12%, by at least 13%, or by at least 14%.

10. The method according to claim 1, wherein the aerodynamic properties of said powder are improved.

11. The method of claim 10, wherein said powder is an inhalable powder.

12. A method of reducing the electrostatics of a powder comprising:
a) obtaining an amorphous powder comprising an active substance and at least one excipient, and
b) exposing said powder over a defined exposure period in controlled manner to a water-containing gas or a solvent-containing gas with a defined relative humidity at a defined temperature.

13. The method according to claim 12, wherein said active substance is a protein.

14. The method according to claim 12, wherein the exposure period is selected such that the excipient crystallises before the active substance.

15. The method according to claim 12, wherein the relative humidity of the water-containing or solvent-containing gas is greater than 30% (w/w), or between 50-60% (w/w).

16. A method of filling powders, comprising treating a powder according to claim 12.

17. The method according to claim 1, wherein the exposure period is at least 8 hours or more, at least 12 hours or more, at least 20 hours or more.

18. The method according to claim 17, wherein the exposure period is 20 hours.

19. The method according to claim 17, wherein the exposure period is 8 hours.

20. The method according to claim 1, wherein the temperature during the exposure time is less than 60° C.

21. The method according to claim 1, wherein the temperature during the exposure time is between −10° C. to 60° C., between 4° C. to 40° C. or between 16° C. and 30° C.

22. The method according to claim 1, wherein the temperature during the exposure time is 4° C., 10° C., ambient temperature or 37° C.

23. The method according to claim 2, wherein said protein is insulin, insulin-like growth factor, human growth hormone (hGH) and other growth factors, tissue plasminogen activator (tPA), erythropoietin (EPO), cytokines, interleukines (IL), IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN)-alpha, -beta, -gamma, -omega or -tau, tumour necrosis factor (TNF), TNF-alpha, beta or gamma, TRAIL, G-CSF, GM-CSF, M-CSF, MCP-1, VEGF, monoclonal, polyclonal, multispecific and single chain antibodies and fragments thereof, Fab, Fab′, F(ab′)₂, Fc and Fc′ fragments, light (L) and heavy (H) immunoglobulin chains and the constant, variable or hypervariable regions thereof or Fv and Fd fragments.

24. A powder obtained by the method of claim 1, wherein said powder has an increased, maintained or minimally reduced flowability (FPF).

25. The powder according to claim 24, wherein said powder has improved aerodynamic properties.

26. The powder according to claim 24, wherein said powder has improved electrostatic properties.

27. The powder according to claim 24, wherein said powder has improved aerodynamic properties and improved electrostatic properties.

28. A powder obtained by the method of claim 1, wherein said powder has increased flowability or increased nano-roughness.
